(19) United States
(12) Patent Application Publication
(10) Pub. No.: US 2010/0256569 A1
(43) Pub. Date: Oct. 7, 2010

(54) MEDICAL INJECTION DEVICE WITH MICRONEEDLES

(75) Inventors: Astrid Cachemaille, Lausanne (CH); Francois Cannehan, Lausanne (CH)

Correspondence Address: NIXON & VANDERHYE, PC, 901 NORTH GLEBE ROAD, 11TH FLOOR, ARLINGTON, VA, 22203, US

(73) Assignee: DEBIOTECH S.A., Lausanne (CH)

(21) Appl. No.: 12/740,023

(22) PCT Filed: Oct. 23, 2008
(86) PCT No.: PCT/IB2008/054370

§ 371 Date: Jun. 15, 2010

(30) Foreign Application Priority Data
Oct. 27, 2007 (IB) PCT/IB2007/054371

(51) Int. Cl. A61M 5/00

(52) U.S. Cl. 604/173

(57) ABSTRACT

The present invention relates to a medical injection device for delivering medium to a patient comprising at least two microneedles, wherein microneedle tips are arranged with a distance.

The present invention relates to a medical injection device, especially for intradermal injection.

An intradermal injection is an injection given to a patient in which the goal is to empty the contents of the syringe between the layers of the skin where it is absorbed. Such an intradermal injection is often used for conducting skin allergy tests and testing for antibody formation.

This is a kind of painful procedure and is usually used only with small amounts of solution.

Intradermal injection devices using needles or microneedles usually lead to a skin bleb during the injection. The reason is that the bolus of substance injected into the dermis is not absorbed or diffused instantaneously. The substance not being absorbed right away results in this bleb.

So even if the method of injection allows a rapid delivery, the substance is absorbed very slowly and therefore the diffusion capacity is the time consuming and therefore determining factor.

It is therefore an object of the present invention to provide a medical injection device which allows a faster and therefore also less painful injection procedure.

Embodiments of the present invention provide a medical injection device for delivering medium to a patient, especially for intradermal injection, comprising at least two microneedles, wherein microneedle tips are arranged with a distance.

A device according to the invention allows the injected medium to cover a larger zone and therefore enhancing the diffusion capacity, instead of being injected by one needle.

Further it could be an advantage if the microneedles are connected to the same dispenser. With an arrangement like this the dispensing unit can be arranged quite easily as well as the assembly will be less complex.

Nevertheless could it be also of advantage in another embodiment of the invention if at least two dispensers are used.

If according to a further preferred embodiment of the invention the microneedles are connected to a substrate the injection of the microneedles could be simplified. Instead of injecting several needles simultaneously the substrate could be applied. This substrate could for example be a chip which could be applied to the patient's skin.

When the microneedles are arranged on a substrate, e.g. a kind of chip, it is further advantageous when the microneedle tips are arranged in a direction away from the substrate. An arrangement like this provides a device where the injected substance covers a zone as large as possible and therefore enhancing its diffusion capacity.

To deliver drugs for example by the medical injection device according to the invention holes could be arranged in the microneedle tips. Preferably these holes and/or the microneedle tips are oriented in a way as to cover a large surface on a patient's skin during an injection.

When using a chip (substrate) including several microneedles, the microneedle orientation and/or the orientation of the holes can be chosen in such a way that all microneedles and/or their corresponding holes or tips “look” towards away from the chip, or some in a given direction and the others in the opposite direction to cover a surface as large as possible.

Such an enhanced diffusion capacity is particularly interesting for an immunization procedure, where when the dentritic cells (Langerhans' cells) are reached, the immune response is most powerful.

If according to a preferred embodiment of the present invention the microneedle tips are oriented in a way as to cover the largest surface on the patient' skin, the diffusion capacity could be further enhanced.

Depending on the application, as e.g. vaccine, patch, . . . , and the quantity of liquid to be delivered to the patient the amount of needles is varied. Different amounts of microneedles could be chosen. Examples are 3 and 5 microneedles in a line and/or 9 and 25 microneedles in a square.

Of course, the invention is not limited to this amount of needles. Every other amount like e.g. even 50, 100 or more could be reasonable. If such a high number is chosen it could be necessary to change the dimensions of the substrate or chip, if used.

Good results could be achieved when the medical injection device includes about three microneedles. This amount of needles leaded with usually used microneedles to a reasonable big area of injection and a reasonable spread supply of the injected substance.

As already mentioned above the medical injection device according to the invention is particularly interesting for an immunization process, where preferably the dentritic cells (Langerhans' cells) are reached resulting in a powerful immune response.

These and other objects, features, aspects and advantages of the present invention will become more apparent from the following detailed description of the present invention when taken in conjunction with the accompanying drawings.

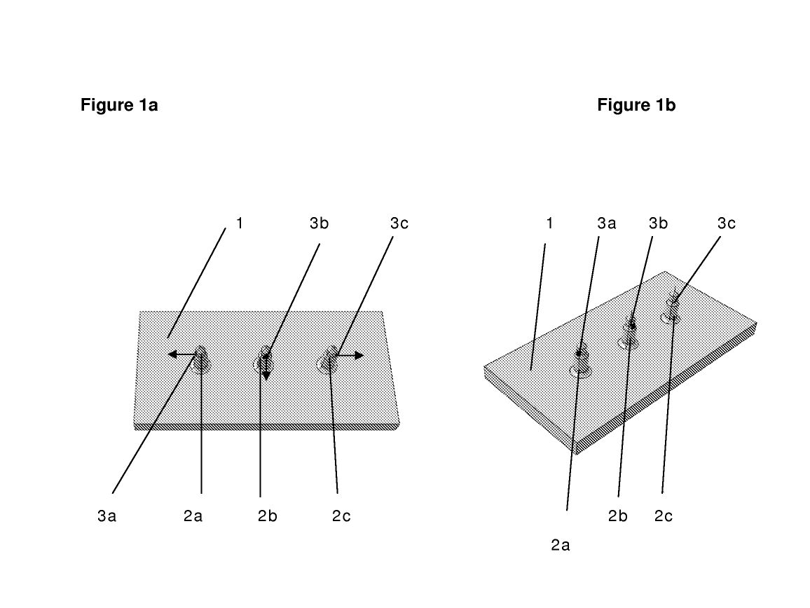

FIGS. 1a and 1b shows a preferred embodiment of a medical injection device according to the invention in different views; and

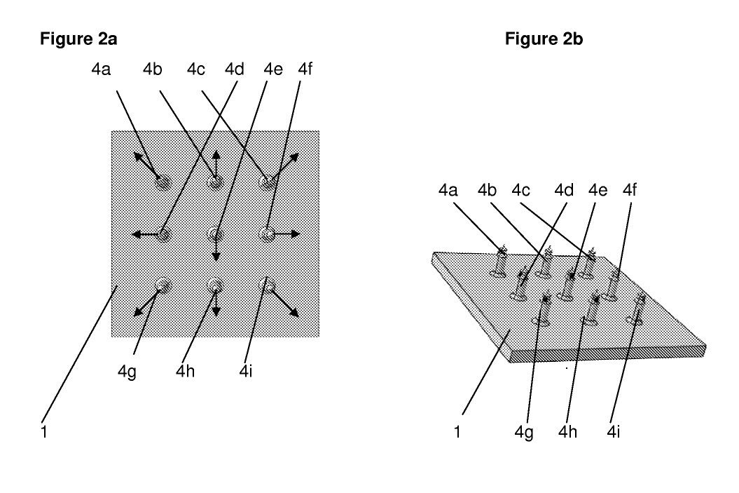

FIGS. 2a and 2 b shows another preferred embodiment of a medical injection device according to the invention in different views.

FIG. 1 shows two different views of a preferred embodiment of a medical injection device according to the invention, wherein on a substrate 1 three microneedles 2a, 2b, 2c are disposed. The microneedles 2a, 2b, 2c are provided with holes 3a, 3b, 3c which orientation is indicated by the flashes in the figure. As can be seen from the figure the orientation of the holes 3a, 3b, 3c and/or the microneedles 2a, 2b, 2c is chosen that way, that every needle is directed away from the chip 1 and the holes 3a, 3b and 3c are oriented in opposite directions so that the injected medium, e.g. drug, covers at least during injection a surface as large as possible.

The same is valid for the embodiment shown in FIGS. 2a and 2b, where 9 microneedles 4a, b, c, d, e, f, g, h, i provided with holes are arranged on a substrate 1. The needles 4a to i are arranged in a rectangular or squared manner. As can be seen from FIG. 2a where the flashes show the orientation of the holes of the microneedles 4a to i, the medium will be delivered in every direction and therefore in an area as big as possible.

1.-11. (canceled)

12. Medical injection device for delivering medium to a patient comprising at least two microneedles connected to a substrate, wherein microneedle tips are provided with holes and are arranged with a distance and in a direction away from said substrate, said holes being oriented along different directions, in a way as to cover a large surface.

13. Medical injection device according to claim 12, wherein the microneedles are connected to a same dispenser.

14. Medical injection device according to claim 13, wherein the microneedle are to at least two dispensers.

15. Medical injection device according to claim 12 and comprising three microneedles.
